(19) United States
(12) Patent Application Publication
(10) Pub. No.: US 2020/0360628 A1
(43) Pub. Date: Nov. 19, 2020

(54) VAPORIZER SYSTEM

(71) Applicant: Wise Ally Holdings Ltd, Hong Kong (CN)

(72) Inventors: Yan Wing MUI, Hong Kong (CN); Tuen Ho CHEUNG, Hong Kong (CN)

(21) Appl. No.: 16/763,555

(22) PCT Filed: Nov. 27, 2017
(86) PCT No.: PCT/CN2017/113026

§ 371 Date: May 13, 2020

(51) Int. Cl. A61M 11/04; A61M 16/06; A61M 15/00; A61M 15/02

(52) U.S. Cl. CPC A61M 11/042; A61M 16/06; A61M 15/0021; A61M 15/003; A61M 15/02; A61M 11/001; A61M 2205/52; A61M 2205/6054; A61M 2205/18; A61M 2205/3331; A61M 2230/42; A61M 2205/3368

(57) ABSTRACT

An apparatus that vaporizes a substance. The apparatus comprises a pod and a vaporizing device that removably connects to the pod. The pod includes a unique identification number and stores the substance to be vaporized and a carrier of the substance. The vaporizing device includes a memory that stores unique identification numbers and temperatures for each of the unique identification numbers, a reader that reads the unique identification number of the pod, and a controller that selects one of temperatures assigned to the unique identification number from the memory, and a heating element that heats the substance and the carrier to the one of the temperatures to vaporize the substance into a vapor for inhalation to a user.

### FIELD OF THE INVENTION

The present invention relates to a method and apparatus or vaporizing a substance to be inhaled by a user.

### BACKGROUND

Medications can be delivered through inhalation directly to the patient's lungs by a medical inhaler or an aerosol therapeutic device. For example, medications may be vaporized by a vaporizer to deliver the active ingredients of the medications to the patient. Efficiency of medication delivery or drug uptake in the nasal and pulmonary tract of the user is a crucial factor.

New methods and systems that provide an efficient, convenient, and safe way for delivering medications via vaporization will assist in advancing technological needs and solving technological problems.

### SUMMARY OF THE INVENTION

One example embodiment is an apparatus that vaporizes a substance. The apparatus comprises a pod and a vaporizing device that removably connects to the pod. The pod includes a unique identification number and stores the substance to be vaporized and a carrier of the substance. The vaporizing device includes a memory that stores unique identification numbers and temperatures for each of the unique identification numbers, a reader that reads the unique identification number of the pod, and a controller that selects one of temperatures assigned to the unique identification number from the memory, and a heating element that heats the substance and the carrier to the one of the temperatures to vaporize the substance into a vapor for inhalation to a user.

Other example embodiments are discussed herein.

### BRIEF DESCRIPTION OF THE DRAWINGS

FIG. 1 shows a block diagram of an apparatus for vaporization in accordance with an example embodiment,

FIG. 2 shows a vaporizer system in accordance with an example embodiment.

FIG. 3 shows a partial sectional view of a vaporizer system in accordance with another example embodiment.

FIG. 4 shows a method that provides a vaporized substance to a user in accordance with another example embodiment.

FIG. 5 shows a block diagram of a vapor-therapy device in accordance with an example embodiment.

FIG. 6 shows a graph of inhalation pressure over time in accordance with an example embodiment.

### DETAILED DESCRIPTION

Example embodiments relate to apparatus and methods that deliver an active ingredient or substance from medications to a user by vaporizing the substance for inhalation to the user.

Traditional Chinese Medicine (TCM) decoction is a time-consuming process. The traditional way of decocting multiple ingredients from the TCM is boiling, which utilizes empirical recipes and requires a long heating process.

Another way of decoction is by using a nebulizer as an aerosol therapy device. Most of the oil-base essence for the aerosol therapy is delivered by ultra-sonic nebulizers. However, nebulization is not efficient, as it delivers aqua-droplets, and the substance to be delivered to the user is suspended in the mist. Due to the droplet size, weight and hydrophobic nature, a great amount of the substance is sedimented along the delivery path and fails to reach the destination.

Example embodiments solve these problems and provide an efficient and convenient way to deliver a vaporized substance via nasal and pulmonary tract by a vaporizer system. The vaporized substance takes a shorter path to reach its destination; for example, drugs for some neural decease and for pulmonary decease will take a short-cut if they are applied on nasal tract.

In one embodiment for example, oil-based essence extracted from TCM or other medications in liquid form is formulated with a carrier that is based on cyclodextrin. The formulated substance is sealed in a pod or a capsule. The substance packed in the pod is heated by a heating element in the vaporizer system and vaporized for inhalation by a user. The vaporizer system allows delivery of a precise amount of vapor to the user.

In one embodiment for example, an apparatus for vaporization comprises a pod and a vaporizing device that removably connects to the pod. The pod stores the substance to be vaporized and a carrier of the substance. The pod is further labeled with a unique identification number, for example, a Radio-frequency identification (RFID) number that corresponds to the ingredient and dosage of the packed substance and its carrier.

The vaporizing device includes a memory that stores unique identification numbers and temperatures for each of the unique identification numbers. Different substances and their carriers packed in the pod have different target temperatures of vaporization.

The vaporizing device further includes a reader that reads the unique identification number of the pod and sends the reading results to a controller of the vaporizing device. The controller selects one of temperatures assigned to the unique identification number from the memory, and commands a heating element to heat the pod to the selected temperature to vaporize the substance into a vapor for inhalation to a user.

In one embodiment for example, the vaporizing device further includes a thermometer that detects a temperature of the pod. A temperature control unit of the vaporizing device adjusts a power of the heating element based on the temperature of the pod detected by the thermometer.

In one embodiment for example, the vaporizing device further includes a pressure sensor that detects a vapor pressure of the vapor inside the pod. A controller turns off the vaporizing device when the detected vapor pressure is out of a predetermined range of pressures.

In one embodiment for example, the vaporizing device further includes a sensor that senses a breathing pace of the user, and a valve that controls a flow of the vapor for inhalation of the user. The valve is turned on and off by the controller of the vaporizing device at a frequency that synchronizes with the breathing pace of the user. As one example, the valve and the heating element of the vaporizing device are simultaneously turned on and off by the controller.

To enhance the delivery efficiency and uptake of the drug, electric charges are added to the target substance or in the flow passage of the vaporized substance. As one example, an ionization unit is disposed at a passage of the vapor of the substance. This unit ionizes the vapor with positive or negative charges to enhance an uptake of the substance into the user.

Example embodiments relate to a vaporizer system that includes a canister having a chamber, a vaporizing device that removably connects to the canister, and a mask that removably connects to the chamber of the canister.

The chamber of the canister receives one or more capsules filled with a liquid substance that is formulated with a carrier based on cyclodextrin for vaporization. The carrier is an amphiphilic carrier that carries both oil and water based substances.

The vaporizing device includes a heating element that heats the liquid substance to a target temperature to vaporize the liquid substance into a vapor. The substance and the carrier of the substance are formulated to be able to vaporize effectively at less than 100° C. to reduce generation of harmful materials.

The substance and the carrier of the substance are formulated to vaporize with particle sizes of 4-10 μm. The particle sizes of 4-10 μm are inhalable sizes but are larger than exhalable sizes, which further enhances the delivery efficiency of the substance to the user.

The mask of the vaporizer system has an air inlet in which the vapor from the chamber flows toward a user and an air outlet in which the user exhales. For example, a mouth or a nose piece provides air inlet and outlet paths. The inhalation cycle is separated from the exhalation cycle by limiting the inhalation path to the nasal tract and the exhalation path to the mouth. The user inhales the vaporized substance through mouth or nose. The mouth piece and nose piece are interchangeable. The vaporizer system can deliver the vaporized substance to the user via nose or mouth by different extension parts.

In one embodiment for example, the vaporizer system further includes a flowmeter to detect a volume of the vapor inhaled by the user. The vaporizer system calculates a consumption dosage of the substance inhaled by the user based on the volume of the vapor inhaled by the user, and records the consumption dosage of the substance inhaled by the user in a memory of the vaporizer system or at a remote server.

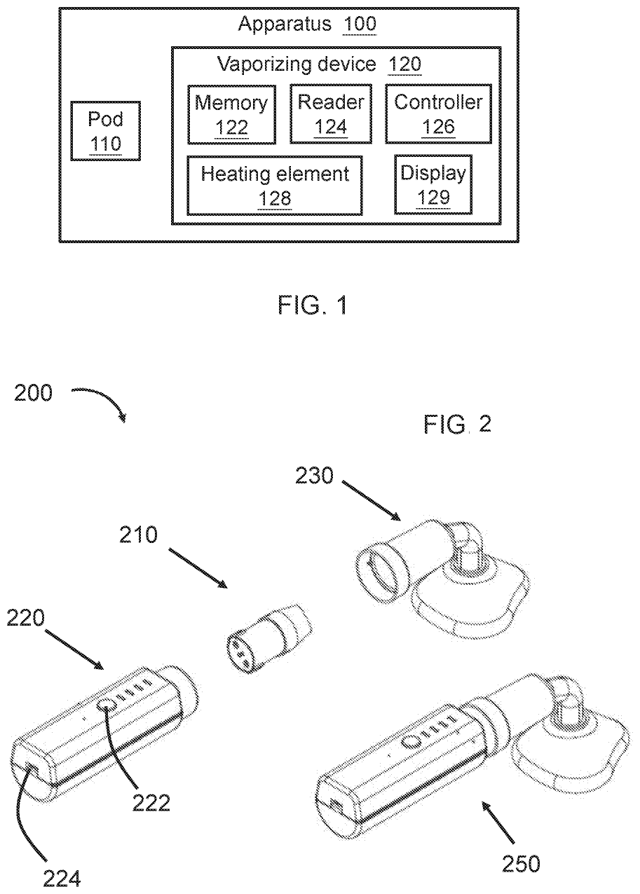

FIG. 1 shows a block diagram of an apparatus for vaporization 100. The apparatus 100 includes a pod 110 and a vaporizing device 120. The vaporizing device 120 includes a memory 122, a reader 124, a controller 126, a heating element 128, and a display 129.

By way of example, the pod 110 stores the substance to be vaporized and a carrier of the substance. The carrier is water-based with both hydrophilic and hydrophobic properties to carry both oil-based and hydrosol substances. The pod is further labeled with a unique identification number, for example, a Radio-frequency identification (RFID) or a OR code that corresponds to the ingredient and dosage of the packed substance and its carrier.

The vaporizing device 120 removably connects to the pod 110 and includes a memory 122 that stores unique identification numbers and temperatures for each of the unique identification numbers. Each combination of substance and its carrier packed in the pod has a temperature of vaporization, and the pod has a unique identification number that reflects the temperature of vaporization for the combination of substance and carrier stored in the pod.

By way of example, three bio-substances, Eucaplytol, Pinene and Limonene, are extracted from natural Eucalyptus oil. These bio-substances are hydrophobic, which means that they are not water soluble. These hydrophobic substances are formulated into a solvent or carrier that includes cyclodextrin. Cyclodextrin has a vaporization temperature less than 100° C. and reduces the vaporization temperature of Eucalyptus Oil from ˜200° C. to less than 100° C. Cyclodextrin further functions as a carrier to carry the three hydrophobic substances via its hydrophobic cavity.

The reader 124 of the vaporizing device 120 reads the unique identification number of the pod when the pod connects with the vaporizing device. The controller 126 has a temperature control unit that receives the unique identification number of the pod sent from the reader. The temperature control unit then selects one of temperatures assigned to the unique identification number from the memory, and commands the heating element 128 to heat the pod to the selected temperature to vaporize the substance into a vapor for inhalation to a user. By way of example, the heating element can be embedded in the main body of the vaporizing device or built inside the pod. The heating element includes positive temperature coefficient thermistor to keep a target temperature.

The display 129 of the vaporizing device 120 displays information related to the status of various elements of the vaporizing device, such as the type of is the substance to be vaporized, the vaporization temperature, and the battery status of a rechargeable battery of the vaporizing device.

FIG. 2 shows a vaporizer system 200 in accordance with an example embodiment. The vaporizer system 200 includes a canister 210 having a chamber, a vaporizing device 220 that removably connects to the canister, and a mask 230 that removably connects to the chamber of the canister 210.

The chamber of the canister 210 receives one or more capsules filled with a liquid substance that is formulated with a carrier based on cyclodextrin for vaporization. The carrier is an amphiphilic carrier that carries both oil and water based substances. The substance and the carrier of the substance are formulated to be able to vaporize effectively at a temperature of less than 100° C. to reduce generation of harmful materials, The vaporizing device 220 has a power button 222 and a power connector 224. The power button 222 allows a user to turn the vaporizing device on an off manually. The power connector 224 charges the rechargeable battery of the vaporizing device or connects the vaporizing device with a power outlet. As one example, the vaporizing device includes operational keys for the user to set parameters such as heating temperature and duration manually. The vaporizing device can also include LED indicators or an alarm buzzer.

The vaporizing device 220 includes a heating element (not shown in FIG. 2) that heats the liquid substance in the capsules to a target temperature to vaporize the liquid substance into a vapor. By way of example, a wick element connects the heating element with the capsule in the chamber of the canister to transfer heat from the heating element to the liquid substance in the capsule and to vaporize the liquid substance to a vapor for inhalation of the user.

The liquid substance and the carrier of the substance are formulated to vaporize into particle sizes of 4-10 μm. The particle sizes of 4-10 μm are inhalable sizes but are larger than exhalable sizes, which enhances the delivery efficiency of the substance to the user.

The mask 230 of the vaporizer system has an air inlet in which the vapor from the chamber flows toward a user and an air outlet in which the user exhales. For example, a mouth or a nose piece provides air inlet and outlet paths. The inhalation cycle is separated from the exhalation cycle by limiting the inhalation path to the nasal tract and the exhalation path to the mouth. The user inhales the vaporized substance by either mouth or nose. The mouth piece and nose piece are interchangeable. The vaporizer system can deliver the vaporized substance to the user via nose or mouth by different extension parts.

When the vaporizer system is in operation, the vaporizer device 220 connects to the canister 210, and further connects to the mask 230, as shown in 250.

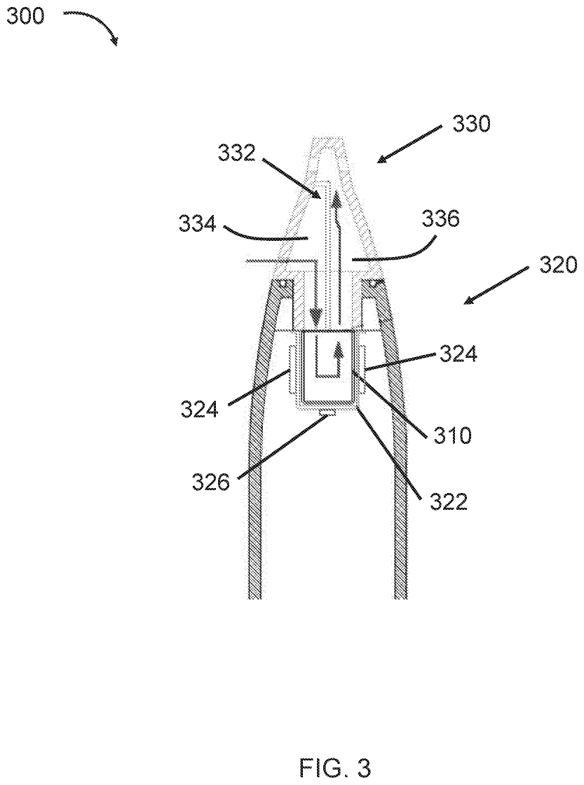

FIG. 3 shows a partial sectional view of a vaporizer system 300. The vaporizer system 300 includes a mask or a mouth piece or a nose piece 330, a canister or a pod 310, and a vaporizing device 320. The vaporizing device 320 further includes a metal surface 322, heating elements 324 and a thermometer 326 that are disposed along the metal surface 322.

By way of example, the canister 310 contacts the metal surface 322 of the vaporizing device 320 to absorb heat. The canister is disposable and is pre-packed with a liquid substance that is formulated with a carrier based on cyclodextrin for vaporization. The carrier is an amphiphilic carrier that carries both oil and water based substances. The substance and the carrier of the substance are formulated to be able to vaporize effectively at a temperature of less than 100° C. to reduce generation of harmful materials.

Heating elements 324 are disposed along the metal surface 322 to heat up the metal surface 322. The metal surface further heats up the canister that contacts the metal surface. The liquid substance in the canister is heated to a target temperature to vaporize into a vapor. The vapor flows into the mask 330 for the user to inhale.

In one embodiment for example, the thermometer 326 of the vaporizing device 320 detects a temperature of the metal surface 322. The vaporizing device adjusts a power of the heating elements based on the temperature of the metal surface detected by the thermometer. For example, when the temperature is higher than a target temperature, the power of the heating elements is reduced to let the temperature reduce to the target temperature.

In one embodiment for example, the vaporizing device further includes a pressure sensor (now shown in FIG. 3) that detects a vapor pressure of the vapor. The pressure sensor is disposed at a passage of the vapor. The vapor pressure drops when the liquid substance in the canister runs out. The vaporizing device shuts itself down when the detected vapor pressure is out of a predetermined range of pressures for security purpose.

In one embodiment for example, the vaporizing device further includes a sensor (now shown in FIG. 3) that senses a breathing pace of the user in order to synchronize the delivery of vaporized substance with the inhalation frequency of the user. A valve communicates with the sensor to control a flow of the vapor to be delivered to the user when the user inhales. The valve is turned on and off by the controller of the vaporizing device at a frequency that synchronizes with the breathing pace of the user. As one example, the valve and the heating element of the vaporizing device are simultaneously turned on and off by the controller.

To enhance the delivery efficiency and uptake of the drug, electric charges are added to the target substance or in the flow passage of the vaporized substance. As one example, an ionization unit is disposed at a passage of the vapor of the substance and that ionizes the vapor with positive or negative charges to enhance an uptake of the substance into the user. As another example, the substance stored in the canister or the pod is electrically charged to enhance an uptake of the substance into the user.

To further enhance the delivery efficiency of the vaporized substance, the liquid substance and the carrier of the substance are formulated to vaporize into particle sizes of 4-10 μm. The particle sizes of 4-10 μm are inhalable sizes but are larger than exhalable sizes, which enhances the delivery efficiency of the substance to the user.

The mask 330 of the vaporizer system has an input chamber 334 and an output chamber 336. The input and output chambers are separated by a divider 332. A carrier gas, such as oxygen, enters the input chamber along the direction of the arrow as shown in FIG. 3 and mix with the vaporized substance to form a mixed gas. The mixed gas flow into the output chamber 336 and further enters the nose or the mouth of the user for inhalation.

By way of example, a mouth or a nose piece provides air inlet and outlet paths, The inhalation cycle is separated from the exhalation cycle by limiting the inhalation path to the nasal tract and the exhalation path to the mouth. The user inhales the vaporized substance by mouth or nose.

In one embodiment for example, the vaporizer system further includes a flowmeter to detect a volume of the vapor inhaled by the user. The vaporizer system calculates a consumption dosage of the substance inhaled by the user based on the volume of the vapor inhaled by the user, and records the consumption dosage of the substance inhaled by the user in a memory of the vaporizer system or at a remote server.

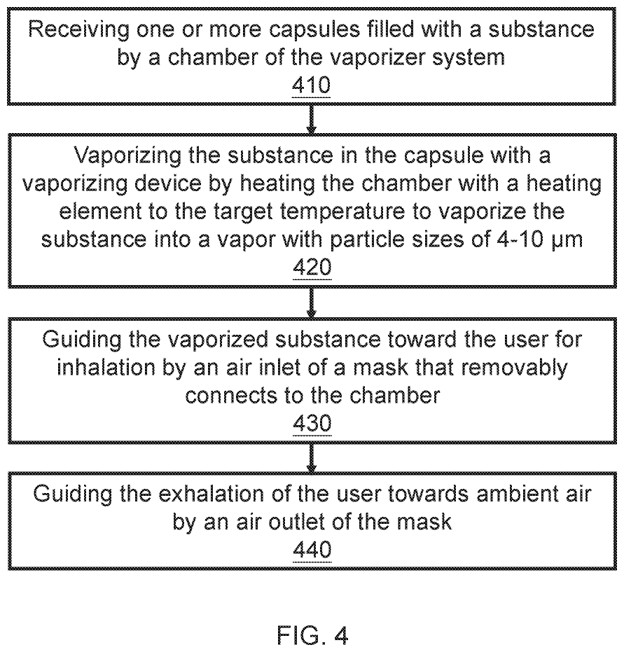

FIG. 4 shows a method that provides a vaporized substance to a user in accordance with an example embodiment.

Block 410 states receiving one or more capsules filled with a substance by a chamber of the vaporizer system.

Consider an example in which a vaporizer system includes a vaporizing device with a chamber, one or more pods or capsules filled with a substance and placed inside the chamber, and a mask that removably connects to the chamber of the vaporizing device of the vaporizer system. The vaporizer system identifies the content of the capsules and carries out a corresponding heating scheme.

The substance is sealed inside the capsule or pod with a metal or foil lid. When the capsule is placed in the chamber, the foil lid is punctuated with holes to allow the vaporized substance to be inhaled.

The capsules are placed inside the chamber of the vaporizing device such that the substance in the capsules are heated up and vaporized into a vapor.

The substance sealed in the capsules is in a liquid form and is formulated with a carrier based on cyclodextrin for vaporization. The substance pre-packed in the capsule includes but not limited to: traditional Chinese medicine concoction, traditional Chinese medicine extract, herbal essence, and 25 aromatherapy essence oil. The carrier is an amphiphilic carrier that carries both oil and water based substances. The substance and the carrier of the substance are formulated to be able to vaporize effectively at a temperature of less than 100° C. to reduce generation of harmful materials.

Block 420 states vaporizing the substance in the capsule with a vaporizing device by heating the chamber with a heating element to the target temperature to vaporize the substance into a vapor with particle sizes of 4-10 μm.

By way of example, the vaporizer system includes a controller that determines a vaporization temperature for the substance and the carrier of the substance that is stored in the capsule, and commands the heating element to heat the capsule to the vaporization temperature to vaporize the substance into a vapor for inhalation to a user.

In one example embodiment, the vaporizing device includes a reader and a memory. The reader reads a unique identification number marked on the capsule. The memory stores unique identification numbers and target temperatures for each of the unique identification numbers, such that the controller determines, from the memory, the target temperature corresponding to the unique identification number of the capsule, and commands the heating element to heat the capsule to the selected temperature to vaporize the substance into a vapor for inhalation to a user.

The heating element of the vaporizer system heats the capsule to vaporize the liquid substance inside the capsule into a vapor. In one example embodiment, a wick element connects the heating element with the capsule in the chamber to transfer heat from the heating element to the substance in the capsule. In another example embodiment, the metal surface transfer heat directly to the substance inside the capsule through the surface of the capsule. In another example embodiment, the liquid substance is poured out of the capsules into the chamber of the vaporizing device to be heated up by the metal surface directly.

The liquid substance and the carrier of the substance are formulated to vaporize into particle sizes of 4-10 μm. The particle sizes of 4-10 μm are inhalable sizes but are larger than exhalable sizes, which enhances the delivery efficiency of the substance to the user.

To further enhance the delivery efficiency and uptake of the drug, electric charges are added to the target substance or in the flow passage of the vaporized substance. As one example, an ionization unit is disposed in the chamber of the vaporizing device and ionizes the vapor with positive or negative charges. As another example, an electroporation process is performed in the air flow passage to add charges to the vapor. As another example, the substance stored in the capsule is formulated with charges before vaporization takes place to enhance an uptake of the substance into the user,

In one embodiment for example, the vaporizer system includes a thermometer to detect a temperature of the chamber that holds the capsule. The vaporizer system adjusts a power of the heating elements when the detected temperature of the chamber is out of a predetermined range of temperatures. For example, when the temperature is higher than a target temperature, the power of the heating elements is reduced to let the temperature reduce to the target temperature. When the temperature is lower than a target temperature, the power of the heating elements is increased to let the temperature rise to the target temperature.

In one embodiment for example, the vaporizer system further includes a pressure sensor that detects a vapor pressure of the vapor inside the chamber of the vaporizing device. The controller of the vaporizer system receives the detected vapor pressure of the chamber and sends out instructions to appropriate elements in the system accordingly. For example, when the detected vapor pressure is out of a predetermined range of pressures, the vaporizing device is shut down for security purpose.

By way of example, the vaporizer system further includes an alarm that signals when a vapor pressure or a temperature of the chamber is out of a predetermined range. The vaporizing device is automatically of manually show down when the alarm signals.

Block 430 states guiding the vaporized substance toward the user for inhalation by an air inlet of a mask that removably connects to the chamber.

The mask of the vaporizer system has an air inlet in which the vapor from the chamber flows toward a user. For example, a nose piece provides air inlet path for the user. The user inhales the vaporized substance through the nose.

In one embodiment for example, the vaporizer system further includes a flowmeter to detect a volume of the vapor inhaled by the user. The flowmeter is disposed at a passage of the vapor of the substance. The vaporizer system calculates a consumption dosage of the substance inhaled by the user based on the volume of the vapor inhaled by the user, and records the consumption dosage of the substance inhaled by the user in a memory of the vaporizer system or at a remote server.

Block 440 states guiding the exhalation of he user towards ambient air by an air outlet of the mask.

The mask of the vaporizer system has an air outlet in which the user exhales. For example, the inhalation cycle is separated from the exhalation cycle by limiting the inhalation path to the nasal tract and the exhalation path to the mouth.

In one embodiment for example, the vaporizer system further includes a sensor that senses a breathing pace of the user in order to synchronize the delivery of vaporized substance with the inhalation frequency of the user. The sensor is disposed at the air inlet of the mask. The controller of the vaporizer system receives the breathing pace from the sensor and controls an open frequency of a valve. The valve is turned on and off by the controller of the vaporizing device at a frequency that synchronizes with the breathing pace of the user. As one example, the valve and the heating element of the vaporizing device are simultaneously turned on and off by the controller. When the valve is open, the vapor flows from the chamber to the air inlet of the mask.

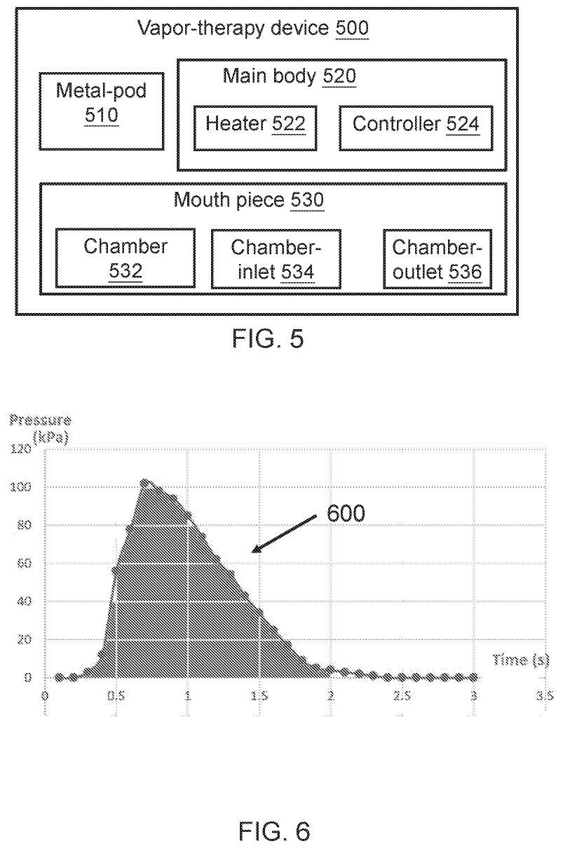

FIG. 5 shows a block diagram of a vapor-therapy device 500 in accordance with an example embodiment. The vapor-therapy device 500 includes a metal-pod 510, a main body 520, and a mouth piece 530. The main body 520 includes a heater 522 and a controller 524. The mouth piece 530 includes a chamber 532, a chamber-inlet 534, and a chamber-outlet 536.

By way of example, the metal-pod 510 stores a medical solution that includes one or more bio-active substances to be vaporized. The medical solution further includes a carrier with a vaporizing point temperature that is less than 65° C. The main body 520 removably connects to the metal-pod. The heater 522 of the main body heats the metal-pod at a temperature that is less than 65° C. The medical solution stored in the metal-pod is heated and vaporized into a vapor.

The controller 524 includes a temperature control unit to regulate or control the temperature of the heater. The chamber 532 of the mouth piece 530 collects the vapor from the metal-pod. Upon the inhalation of the user, the air flows through the chamber-inlet 534 of the mouth piece, mixes with the vapor in in the chamber 532 and finally flows through the chamber-outlet 536 towards a user such that the bio-active substances are delivered to the user.

The controller 524 has a temperature control unit that commands the heater 522 to heat the metal-pod 510 to a temperature to vaporize the medical solution into a vapor for inhalation to a user. By way of example, the heater is embedded in or included with the main body of the vapor-therapy device and includes a positive temperature coefficient thermistor to maintain a target temperature.

In one example embodiment, the metal-pod further includes an amphiphilic solvent that functions as a carrier to bind the bio-active substance. The bioactive substance can be either hydrophilic or hydrophobic. As one example, the amphiphilic solvent carries a plurality of bio-substances with herbal ingredients that are hydrophilic or hydrophobic.

The solvent or carrier is an amphiphilic carrier that carries both oil and water based substances. The substance and the carrier of the substance are formulated to vaporize effectively at a temperature of less than 65° C. to reduce generation of harmful materials. Due to the low vaporizing point temperature of the carrier, minimal heat energy is needed to create sufficient surface vapor-pressure for vaporization. The vaporization process does not need extra mechanical or kinetic force, which are usually needed for conventional ultrasonic vaporizers.

By way of example, the medical solution is vaporized into the vapor in a molecular-level particle size of 4-10 μm. The particle size of 4-10 μm is inhalable but is larger than exhalable sizes, which enhances the delivery efficiency of the substance to the user. By contrast, conventional ultra-sonic nebulizers deliver aqua-droplets, and the substance to be delivered to the user is suspended in the mist. Due to the droplet size, weight and hydrophobic nature, a great amount of the substance is sedimented along the delivery path and fails to reach the destination with conventional nebulizers and drug delivery devices.

FIG. 6 shows a graph 600 of inhalation pressure over time in accordance with an example embodiment. The X-axis shows time, and the Y-axis shows the inhalation pressure.

Consider an example in which a vapor-therapy device delivers vaporized substances to a user through a mouth piece. The vapor-therapy device includes a metal-pod, a main body and a mouth piece. The metal-pod stores a medical solution that includes one or more bio-active substances to be vaporized. The main body includes a heater to heat the metal-pod and a controller to control the temperature of the heater. The medical solution stored in the metal-pod is heated by the heater and vaporized into a vapor.

The mouth piece includes a chamber, a chamber-inlet 534, and a chamber-outlet 536. The chamber of the mouth piece collects the vapor from the metal-pod. The vapor flows in the chamber-outlet of the mouth piece towards a user to deliver the bio-active substances to the user.

In one example embodiment, the chamber of the mouth piece includes or is built with an equilibrium volume. The equilibrium volume maintains the medical solution at a vapor state at a temperature less than 65° C. with saturated bio-active substances. The equilibrium volume of the chamber is determined by:

$${V}_{eql} = n {\int }_{t = 0}^{t = {t}_{interval}} \frac{T ( t )}{P ( t )} dt ,$$

where V_{eql} is the equilibrium volume of the chamber, tᵢₙₜₑᵣᵥₐₗ is an inhalation interval of the user, T(t) is a change of temperature during the inhalation interval of the user, and P(t) is a change of vapor pressure in the chamber of the mouth piece during the inhalation interval.

In one example embodiment, the chamber-inlet of the mouth piece further includes a pressure sensor that detects an inhalation pressure of the user and an inhalation duration of the user. The detected duration and pressure will be further deduced into the inhalation volume of the user, represented by:

$${V}_{inhale} = n {\int }_{t = 0}^{t = {t}_{inhale}} P ( t ) dt ,$$

where Vᵢₙₕₐₗₑ is the inhalation volume of the user, P(t) is a change of inhalation pressure during the inhalation, as shown in curve 600 of FIG. 6, and tᵢₙₕₐₗₑ is an inhalation duration of the user.

The controller of the vapor-therapy device adjusts the temperature of the heater, based on an inhalation capacity of the user and a volume of the chamber of the mouth piece, to achieve a target in-take dosage of the user. For example, the ratio of V_{eql}/Vᵢₙₕₐₗₑ can be correlated to achieve a target concentration of the in-take dosage of the user. If this ratio exceeds the design target, the difference is sent to the controller of the vapor-therapy device to adjust the temperature of the heater and feed a right proportion of turn-over dosage for each inhalation, according to the inhalation capacity of each individual user.

As used herein, “vaporization” is a process that vaporizes active ingredients or substances of a material for the purpose of inhalation.

to As used herein, a “pod” or “canister” is a container used for storing solid, liquid or gas materials.

The methods and apparatus in accordance with example embodiments are provided as examples, and examples from one method or apparatus should not be construed to limit examples from another method or apparatus. Further, methods and apparatus discussed within different figures can be added to or exchanged with methods and apparatus in other figures. Further yet, specific numerical data values (such as specific quantities, numbers, categories, etc.) or other specific information should be interpreted as illustrative for discussing example embodiments.

What is claimed is:

1. An apparatus that vaporizes a substance, the apparatus comprising:
a pod that includes a unique identification number and that stores the substance to be vaporized and a carrier of the substance; and
a vaporizing device that removably connects to the pod and that includes:
a memory that stores unique identification numbers and temperatures for each of the unique identification numbers;
a reader that reads the unique identification number of the pod;
a controller that includes a temperature control unit that selects, from the memory, one of temperatures assigned to the unique identification number; and
a heating element that heats the substance and the carrier to the one of the temperatures to vaporize the substance into a vapor for inhalation to a user.

2. The apparatus of claim 1 further comprising:
a thermometer that detects a temperature of the pod and sends the detected temperature to the temperature control unit, wherein the temperature control unit adjusts a power of the heating element based on the temperature of the pod detected by the thermometer.

3. The apparatus of claim 1 further comprising:
a pressure sensor that detects a vapor pressure of the vapor inside the pod and sends the detected vapor pressure to the controller of the apparatus, wherein the controller turns off the apparatus when the detected vapor pressure is out of a predetermined range of pressures.

4. The apparatus of claim 1, wherein the vaporizing device vaporizes the substance into sizes of 4-10 μm at a temperature that is less than 100° C.

5. The apparatus of claim 1 further comprising:
a sensor that senses a breathing pace of the user and sends the breathing pace to the controller of the apparatus; and
a valve that controls a flow of the vapor for inhalation of the user,
wherein the valve is turned on and off by the controller at a frequency that synchronizes with the breathing pace of the user.

6. The apparatus of claim 1 further comprising:
a valve that controls an output of the vapor for inhalation of the user,
wherein the valve and the heating element are simultaneously turned on and off by the controller of the apparatus at a predetermined frequency.

7. The apparatus of claim 1 further comprising:
an ionization unit disposed at a passage of the vapor of the substance and that ionizes the vapor with positive or negative charges to enhance an uptake of the substance into the user.

8. The apparatus of claim 1 further comprising:
a recorder that records a consumption dosage of the substance inhaled by the user based on a reading from a flowmeter that is disposed at a passage of the vapor of the substance.

9. A vaporizer system, comprising:
a canister having a chamber to receive one or more capsules filled with a liquid substance that is formulated with a carrier based on cyclodextrin for vaporization;
a vaporizing device that removably connects to the canister and that includes:
a heating element that heats the liquid substance to a target temperature to vaporize the liquid substance into a vapor with particle sizes of 4-10 μm; and
a controller that determines the target temperature of the liquid substance and turns the heating element on and off; and
a mask that removably connects to the chamber of the canister and has an air inlet in which the vapor from the chamber flows toward a user and an air outlet in which the user exhales.

10. The vaporizer system of claim 9, wherein each of the capsules is assigned with a unique identification number, and wherein the vaporizing device further comprising:
a reader that reads the unique identification number from the capsule that is placed into the chamber of the canister; and
a memory that stores unique identification numbers and target temperatures for each of the unique identification numbers such that the controller determines, from the memory, the target temperature corresponding to the unique identification number of the capsule.

11. The vaporizer system of claim 9, further comprising:
a thermometer that detects a temperature of the chamber of the canister and sends the detected temperature to the controller of the vaporizer system, wherein the controller adjusts a power of the heating element based on the temperature of the chamber detected by the thermometer.

12. The vaporizer system of claim 9, further comprising:
a pressure sensor that detects a vapor pressure of the vapor inside the chamber and sends the detected vapor pressure to the controller of the vaporizing device, wherein the controller turns off the vaporizing device when the detected vapor pressure is out of a predetermined range of pressures.

13. The vaporizer system of claim 9 further comprising:
a sensor disposed at the air inlet of the mask that senses a breathing pace of the user and sends the breathing pace to the controller of the vaporizing device; and
a valve that controls an output flow of the vapor,
wherein the valve is turned on and off by the controller at a frequency that synchronizes with the breathing pace of the user.

14. The vaporizer system of claim 9 further comprising:
a valve that controls an output of the vapor for inhalation of the user,
wherein the valve and the heating element are simultaneously turned on and off by the controller of the vaporizing device at a predetermined frequency.

15. The vaporizer system of claim 9, wherein the liquid substance in the capsule is electrically charged to enhance an uptake of the liquid substance into the user.

16. The vaporizer system of claim 9 further comprising:
a wick element that connects the heating element with the capsule in the chamber of the canister.

17. A method executed by a vaporizer system that provides a vaporized substance to a user for inhalation, the method comprising:
receiving, by a chamber of the vaporizer system, one or more capsules filled with a substance, wherein the substance is formulated with a carrier such that the substance can be vaporized at a temperature that is less than 100° C.;
vaporizing, by a vaporizing device that removably connects to the chamber of the vaporizer system, the substance in the capsule by:
determining, by a controller of the vaporizer system, a target temperature that vaporizes the substance with the carrier; and
heating the chamber with a heating element to the target temperature to vaporize the substance into a vapor with particle sizes of 4-10 μm;
guiding, by an air inlet of a mask that removably connects to the chamber, the vaporized substance toward the user for inhalation; and
guiding, by an air outlet of the mask, the exhalation of the user towards ambient air.

18. The method of claim 17, wherein each of the capsules is assigned with a unique identification number, and wherein the vaporizing device further comprising:
a reader that reads the unique identification number from the capsule that is placed into the chamber of the canister; and
a memory that stores unique identification numbers and target temperatures for each of the unique identification numbers, such that the controller determines, from the memory, the target temperature corresponding to the unique identification number of the capsule.

19. The method of claim 17 further comprising:
detecting, by a thermometer, a temperature of the chamber;
receiving, by the controller of the vaporizing device of the vaporizer system, the detected temperature of the chamber; and
adjusting, by the controller of the vaporizing device of the vaporizer system, a power of the heating element when the detected temperature of the chamber is out of a predetermined range of temperatures.

20. The method of claim 17 further comprising:
detecting, by a pressure sensor, a vapor pressure of he vapor inside the chamber;
receiving, by the controller of the vaporizing device of the vaporizer system, the detected vapor pressure of the chamber; and
shutting down the vaporizing device when the detected vapor pressure is out of a predetermined range of pressures.

21. The method of claim 17, further comprising:
detecting, by a sensor disposed at the air inlet of the mask, a breathing pace of the user;
receiving, by the controller of the vaporizing device of he vaporizer system, the breathing pace of the user; and
synchronizing, by the controller of the vaporizing device, an open frequency of a valve with the breathing pace of the user,
wherein the vapor flows from the chamber to the air inlet of the mask when the valve is open.

22. The method of claim 17, wherein the vaporizer system further comprising:
a valve that controls an output of the vapor for inhalation of the user,
wherein the valve and the heating element are simultaneously turned on and off by the controller of the vaporizing device at a predetermined frequency.

23. The method of claim 17, wherein the substance in the capsule is electrically charged to enhance an uptake of the substance into the user.

24. The method of claim 17, wherein the vaporizer system further including an ionization unit disposed at a passage of the vapor and that ionizes the vapor with positive or negative charges to enhance an uptake of the substance into the user.

25. The method of claim 17, wherein the vaporizer system further includes an alarm that signals when a vapor pressure or a temperature of the chamber is out of a predetermined range.

25. The method of claim 17, further comprising:
detecting, by a flowmeter disposed at a passage of he vapor of the substance, a volume of the vapor inhaled by the user:
calculating, by the vaporizer system, a consumption dosage of the substance inhaled by the user based on the volume of the vapor inhaled by the user; and
recording, at a server, the consumption dosage of the substance inhaled by the user.

26. A vapor-therapy device that vaporizes a medical solution, comprising:
a metal-pod that stores the medical solution that includes a bio-active substance;
a main body that removably connects to the metal-pod and that includes:
a heater that heats the metal-pod at a temperature that is less than 65° C., such that the medical solution stored in the metal-pod is heated and vaporized into a vapor; and
a controller that includes a temperature control unit to regulate the temperature of the heater; and
a mouth piece that includes a chamber to collect the vapor, a chamber-inlet in which air flows in and mixes with the vapor, and further flows through a chamber-outlet toward a user to deliver the bio-active substance to the user.

27. The vapor-therapy device of claim 26, wherein the metal-pod further includes an amphiphilic solvent that functions as a carrier to bind the bio-active substance, wherein the bio-active substance is hydrophilic or hydrophobic.

28. The vapor-therapy device of claim 26, wherein the medical solution further includes a carrier with a vaporizing point temperature that is less than 65° C.

29. The vapor-therapy device of claim 26, wherein the medical solution is vaporized into the vapor in a molecular-level particle size.

30. The vapor-therapy device of claim 26, wherein the metal-pod further includes a solvent that functions as a carrier that carries hydrophobic and hydrophilic substances in vapor state.

31. The vapor-therapy device of claim 26, wherein the metal-pod further includes a solvent that functions as a carrier that carries a plurality of bio-substances with herbal ingredients that are hydrophilic or hydrophobic.

32. The vapor-therapy device of claim 26, wherein the chamber of the mouth piece is built with an equilibrium volume, and wherein the equilibrium volume maintains the medical solution at a vapor state with saturated bio-active substances.

33. The vapor-therapy device of claim 26, wherein the chamber-inlet of the mouth piece further includes a pressure sensor that detects an inhalation pressure of the user and an inhalation duration of the user.

34. The vapor-therapy device of claim 26, wherein the controller adjusts the temperature of the heater, based on an inhalation capacity of the user and a volume of the chamber of the mouth piece, to achieve a target in-take dosage of the user.
